(19) United States
(12) Patent Application Publication
(10) Pub. No.: US 2014/0004090 A1
(43) Pub. Date: Jan. 2, 2014

(54) METHOD OF MODIFYING BACTERIA

(71) Applicants: Alejandra Vasquez, Raa (SE); Tobias Olofsson, Raa (SE)

(72) Inventors: Alejandra Vasquez, Raa (SE); Tobias Olofsson, Raa (SE)

(73) Assignee: CONCELLAE AB, Helsingborg (SE)

(21) Appl. No.: 13/981,168

(22) PCT Filed: Jan. 24, 2012
(86) PCT No.: PCT/SE12/50063

§ 371 Date: Sep. 11, 2013

Related U.S. Application Data
(60) Provisional application No. 61/435,638, filed on Jan. 24, 2011.

(30) Foreign Application Priority Data
Jan. 24, 2011 (SE) 1150041-0

(51) Int. Cl. C12N 1/20; A23L 1/30; A23K 1/00

(52) U.S. Cl. CPC C12N 1/20; A23K 1/009; A23L 1/3014
USPC 424/93.45; 435/252.9; 435/252.1; 435/253.6; 424/93.4

(57) ABSTRACT

A method of enhancing the antimicrobial properties of a bacterial strain, comprising cultivation of the bacterial strain in a medium comprising water, at least one component chosen from bee pollen, pollen, and bee bread, and optionally honey and/or nectar is described. The bacterial strain has the ability to be viable for at least 8 days in a 65% by weight sugar solution, preferably at least 8 days in a 70% by weight sugar solution.

### FIELD OF THE INVENTION

The present invention relates to a method of enhancing the antimicrobial properties of a bacterial strain, comprising cultivation of the bacterial strain in a medium comprising water, at least one component chosen from bee pollen, and bee bread, and optionally honey and/or nectar.

The present invention also relates to a modified bacterial strain produced using the method of the invention, and a pharmaceutical composition or a feed or food product comprising the modified bacterial strain.

Further, the present invention also relates to a cultivation medium for lactic acid bacteria and a symbiotic comprising the cultivation medium.

Cultivation of a bacterial strain in a medium according to the present invention enhances the production of antimicrobial products of honey bee specific lactic acid bacteria (LAB) against pathogens and food spoiling microorganisms connected to humans, honey bees and their larvae. The bacteria with enhanced production of antimicrobial substances are preferably honey bee isolated Lactobacillus and Bifidobacterium strains from the honey producing tract of honey bees and stingless bees (referred to as “bees”). The bacterial strains according to the present invention further have the ability to be viable for at least 8 days in a 65% by weight sugar solution, preferably at least 8 days in a 70% by weight sugar solution. The growth media according to the present invention is organic and gives the bacterial strains unique properties rendering them useful in many products such as in functional food, probiotics, synbiotics, prebiotics, beverage products, feed products and medical products. The growth media also increases their growth rate significant.

### BACKGROUND OF THE INVENTION

Bees live in symbiosis with a flora of lactic acid bacteria (LAB) of the genera Lactobacillus and Bifidobacterium in their honey producing tract. All the different members have evolved together and live side by side in a symbiosis with the honey bee. The honey bee getting protection against bee diseases, larval diseases and microorganisms that can spoil their food stuff as nectar, honey, and bee pollen and bee bread. It was first discovered by Olofsson and Vasquez and applications of these bacteria are protected in a patent application (International Application No.: PCT/SE2008/000303).

Honeybees use pollen as a source of vitamins, proteins, fatty acids, lipids, sterols, minerals and carbohydrates for their nutrition. Bees collect pollen and store it in the colony as bee bread which is then consumed by adult bees and it is also fed to the larvae. Bee bread is produced by lactic acid fermentation with the help of their honey stomach LAB.

Initially bee bread is composed of bee pollen that contains pollen, flower nectar or honey, the honey stomach LAB and secretions from the bee's salivary glands. The bee pollen is collected by foraging bees during their visits to flowers and transported on their hind legs in a specialized pollen basket back to the hive. It is then packed into cells of the brood comb by house bees that eventually seal it with a drop of honey. After two weeks it is chemically changed by what is believed to be a natural fermentation of the LAB. After the two week fermentation period the bee bread is preserved by the fermentation and will last for many months. The nutrients are now available for not only the bee larvae but also for the LAB.

The LAB flora helps the bees to produce their food honey, bee pollen and bee bread. They also help the bees to prevent spoilage of this food from other bacteria, yeast and mould. Furthermore the LAB helps the bees to fight honey bee diseases and honey bee larval diseases.

The bee specific LAB live and multiply in the honey stomach and in the bee food products and the bee food products are the food for these bacteria. From self-obtained novel knowledge we now know that the LAB in the honey stomach also needs all the nutrients from pollen. Pollen grains have a hard coat that protects them from almost any outer physical stress making the nutrients inside hard to access for the LAB. In bee bread, the nutrients from pollen are made available by the fermentation of the bee LAB. When bees consume bee bread it passes through the honey stomach and now even the LAB in the honey stomach can utilize the nutrients from the pollen.

Notably, very few bees eat bee bread and the honey stomach LAB most often need to get the nutrients from pollen in another way than from the bee bread. When nectar is collected from flowers by bees during honey production a large amount of pollen from the same flower follow the nectar to the honey stomach. The nectar contains a high concentration of sugars but also water. We know now that some of the pollen grains burst in this mix of sugar and water in the honey stomach due to osmotic shock, making the pollen nutrients available for the LAB.

LAB are normally cultivated in a laboratory on well-known commercial bacterial media plain or modified such as Rogosa, MRS, API, Tomato juice, LCM etc. These media are in one way or the other composed in a synthetic matter.

The bacterial growth, growth rate, production of antimicrobial substances and other essential substances of a bee specific LAB flora are markedly enhanced when grown on their natural and organic media in comparison with a commercial growth media. It is an important issue to grow these bacteria in proper media before using them in products against human pathogens and bee pathogens and in products for preservation of different food and feed.

Not only the classical production of LAB antimicrobial substances is enhanced but also other substances and nutrients that enhance both the defence system among the honey stomach LAB and the defence system (immune system) among bees and humans and the combination of both since the LAB lives in symbiosis within their host.

It is essential nutrients for bees and their larvae and humans like vitamins. It is substances that make the LAB cell wall more rigid which is an advantage in their defence system. It is substances that make the LAB stick to surfaces were they can form biofilm which enhances their defending abilities. It is production of substances that works as a camouflage making them “invisible” for the host immune system. Finally, the enhanced bacterial growth and growth rate are very important factors regarding microbial defence numerically.

### SUMMARY OF THE INVENTION

The present invention relates to a method of enhancing the antimicrobial properties of a bacterial strain, comprising cultivation of the bacterial strain in a medium comprising water, at least one component chosen from bee pollen, pollen, and bee bread, and optionally honey and/or nectar, wherein the bacterial strain has the ability to be viable for at least 8 days in a 65% by weight sugar solution, preferably at least 8 days in a 70% by weight sugar solution.

According to another aspect, the bacterial strain of the present invention is chosen from the genus Lactobacillus and the genus Bifidobacterium.

According to yet another aspect, the bacterial strain can be isolated naturally from the honey producing tract of at least one bee, wherein the honey producing tract of a bee consists of the trunk, mouth, esophagus and honey sac.

In another aspect, the bacterial strain is chosen from Lactobacillus strain Biut2 (LMG P-24094), Lactobacillus strain Hma2 (LMG P-24093), Lactobacillus strain Hma8 (LMG P-24092), Lactobacillus strain Bma5 (LMG P-24090), Lactobacillus strain Hon2 (LMG P-24091) said strains being deposited at BCCM/LMG Bacteria Collection in Belgium on 3 Apr. 2007, Bifidobacterium strain Bin7 (LMG P-23986), Bifidobacterium strain Hma3 (LMG P-23983), Bifidobacterium strain Bing (LMG P-23984), Bifidobacterium strain Bma6 (LMG P-23985) and Lactobacillus kunkeei Fhon2 (LMG P-23987), said strains being deposited at BCCM/LMG Bacteria Collection in Belgium on 15 Jan. 2007 and Hma11 (LMG P-24612) deposited at BCCM/LMG Bacteria Collection in Belgium on Apr. 28, 2008.

According to another aspect, the bacterial strain originates from human or animal.

The present invention further relates to a modified bacterial strain produced according to the method as described above.

According to one aspect, the present invention relates to a modified bacterial strain produced according to the method above for use in the treatment and/or prevention of throat infections.

According to a further aspect, the present invention relates to a modified bacterial strain produced according to the method above for use in the treatment of a wound.

In one aspect, the treatment of a wound includes promotion of wound healing.

The present invention further relates to a pharmaceutical composition comprising a modified bacterial strain according to the invention and a pharmaceutically acceptable carrier and/or diluent.

Further, the present invention relates to a feed or food product comprising a modified bacterial strain according to the above.

The present invention relates, in one aspect, to a cultivation medium for lactic acid bacteria comprising at least one component chosen from bee pollen, pollen, and bee bread, and another component chosen from water, and optionally honey and/or nectar.

In one aspect, the bee pollen, pollen, bee bread, nectar and honey have been obtained from bee pollen grains.

The present invention relates, in another aspect, to a synbiotic comprising a cultivation medium according to the above and at least one strain of lactic acid bacteria.

In one aspect, the at least one strain of lactic acid bacteria is chosen from the genus Lactobacillus and the genus Bifidobacterium.

The present invention relates in yet another aspect to a feed or food product comprising a synbiotic according to the above. The present invention relates in another aspect to a pharmaceutical composition comprising a synbiotic according to the present invention and a pharmaceutically acceptable carrier and/or diluent.

The present invention relates in another aspect to a synbiotic or a food or feed product or a pharmaceutical composition for use in treating and/or preventing infections and/or gastrointestinal diseases

The present invention relates in another aspect to the use of a synbiotic or a food or feed product a pharmaceutical composition for the manufacture of a composition for treating and/or preventing infections and/or gastrointestinal diseases.

The present invention relates in another aspect to the use of a cultivation medium according to present invention for cultivation of lactic acid bacteria chosen from Bifidobacterium or Lactobacillus, preferably isolated from a bee, and for production of antimicrobial substances and/or nutrients.

The present invention relates in a yet another aspect to a method for obtaining a cultivation medium for lactic acid bacteria comprising:
a) providing bee pollen comprising bee pollen grains;
b) mixing the bee pollen with water to provide a mixture;
c) letting the mixture stand at room temperature until the bee bollen grains burst due to osmotic shock;
d) filtering off large bee pollen particles and centrifuging the mixture; and
e) optionally filtering off microorganisms originating from flowers.

The invention according to the present invention relates to a method of enhancing the antimicrobial properties of a bacterial strain, which strain has the ability to be viable for at least 8 days in a 65% by weight sugar solution, preferably at least 8 days in a 70% by weight sugar solution. The antimicrobial properties and growth rates of bee specific LAB which when grown on the media constituting bee food products are significantly enhanced. The bacterial strains show unique properties rendering them useful in many products such as in food products, beverage products, feed products and medical products. The isolated bacterial strains grow quickly and can efficiently combat other microorganisms when they have access to the right nutrients from the bee food products that they are naturally adapted to. They are especially efficient against organisms that are well known to spoil human food, bee food and feed products and against organisms causing infections among humans and bees. Furthermore the honey stomach LAB, when grown on the novel media, produce substances and nutrients that enhance both the antimicrobial defence system among the LAB and the defence system (immune system) among bees and humans. Notably, the production of antimicrobial substances is superially triggered by the presence of microorganisms following the bee pollen like other bacteria and yeast and molds. The outcome is bacteria or bacterial products well suited to be used in food products, beverage products, feed products and medical products especially those containing honey, bee bread, bee pollen or pollen. These products have unique health promoting properties.

In one aspect, the invention relates to a method for the manufacturing of a novel growth media for bee specific lactic acid bacteria that enhance their growth and production of antimicrobial properties. The said media containing honey, bee bread, bee pollen, pollen and water, using at least one of the mentioned bee products as an ingredient.

In another aspect, the invention relates to the use of the product described in the first aspect of the invention for the production of a bacterial culture and its metabolites using at least one lactic acid bacterial strain from the honey producing tract of at least one bee or from fresh honey, bee pollen or bee bread produced by at least one bee.

In another aspect the invention relates to the use of the products described above combined or to the separate use of the products, in a food product, feed product or medical product.

Further advantages and objects with the present invention will be described in more detail, inter alia with reference to the accompanying drawings.

### BRIEF DESCRIPTION OF THE DRAWINGS

FIG. 1. Average growth of bee specific lactic acid bacteria on the commercial media MRS enhanced with fructose and L-cystein in comparison with the novel “bee product” media. Cultivation on the “bee product” media (red) gives a 3 times faster growth and a 3 times larger end product. (X-axis: time (min), Y-axis: OD-measurement.)

### DETAILED DESCRIPTION OF THE INVENTION

The present invention relates to a method of enhancing the antimicrobial properties of a bacterial strain, comprising cultivation of the bacterial strain in a medium comprising water, at least one component chosen from bee pollen, pollen, and bee bread, and optionally honey and/or nectar, wherein the bacterial strain has the ability to be viable for at least 8 days in a 65% by weight sugar solution, preferably at least 8 days in a 70% by weight sugar solution.

In one embodiment of the invention a cultivation medium for lactic acid bacteria is provided comprising at least one component chosen from bee pollen, pollen, and bee bread, and another component chosen from water, and optionally honey and/or nectar, wherein said bee pollen, pollen, bee bread, nectar and honey have been obtained from bee pollen grains. In another embodiment a synbiotic is provided as stated above, wherein said at least one strain of lactic acid bacteria is chosen from the genus Lactobacillus and genus Bifidobacterium, preferably wherein said at least one strain of lactic acid bacteria chosen from the genus Lactobacillus and genus Bifidobacterium has been isolated from a bee, preferably of the genus Apis.

In another embodiment a synbiotic is provided, wherein said lactic acid bacteria represents the lactic acid bacteria flora of the stomach of at least one bee species.

In another embodiment a synbiotic is provided, wherein said lactic acid bactera is chosen from Lactobacillus strain Biut2 (LMG P-24094), Lactobacillus strain Hma2 (LMG P-24093), Lactobacillus strain Hma8 (LMG P-24092), Lactobacillus strain Bma5 (LMG P-24090), Lactobacillus strain Hon2 (LMG P-24091) said strains being deposited at BCCM/LMG Bacteria Collection in Belgium on 3 Apr. 2007, Bifidobacterium strain Bin7 (LMG P-23986), Bifidobacterium strain Hma3 (LMG P-23983), Bifidobacterium strain Bin2 (LMG P-23984), Bifidobacterium strain Bma6 (LMG P-23985) and Lactobacillus kunkeei Fhon2 (LMG P-23987), said strains being deposited at BCCM/LMG Bacteria Collection in Belgium on 15 Jan. 2007 and Hma11 (LMG P-24612) deposited at BCCM/LMG Bacteria Collection in Belgium on Apr. 28, 2008.

In another embodiment of the invention a synbiotic is provided, wherein said synbiotic is freeze-dried.

In another embodiment of the invention a method for obtaining a cultivation medium for lactic acid bacteria is provided comprising:
a) providing bee pollen comprising bee pollen grains;
b) mixing the bee pollen with water to provide a mixture;
c) letting the mixture stand at room temperature until the bee pollen grains burst due to osmotic shock;
d) filtering off large bee pollen particles and centrifuging the mixture; and
e) optionally filtering off microorganisms originating from flowers.

In another embodiment a cultivation medium obtainable by the above mentioned method is provided.

### Definitions

In the context of the present application and invention, the following definitions apply:

The term “pollen” means a fine to coarse powder containing the microgametophytes of seed plants, which produce the male gametes (sperm cells).

The term “bee pollen” means pollen combined by a bee with honey or nectar or other sugar source.

The term “bee bread” means bee pollen fermented by microorganisms in the bee hive.

The term “microorganisms” means both bacteria and fungi such as yeast or mould.

The term “honey” means the sweet, viscous liquid produced in the honey producing tract of various bees from the nectar of flowers.

The term “honey producing tract” means the trunk, mouth, esophagus and honey sac (stomach) of a honey bee.

The term “fresh honey” means honey not older than three days after the gathering of nectar by a honey bee to the beehive. Furthermore, “fresh honey” has got water content above about 18% and resides in not yet wax sealed cells. In contrast, ripe honey has got water content below about 18%.

The term “antimicrobial properties” relates to antibacterial substances produced by bacteria displaying a bactericidal or fungicidal mode of action towards food spoilage and pathogenic microorganisms.

The term “CFU” means colony-forming unit.

The term “sugar source” means in general a sweet soluble disaccharide or small oligosaccharide carbohydrate. Examples of sugar sources are honey, sugar, glucose, fructose, sucrose and maltose.

The term “lactic acid bacteria, LAB” relates to bacteria producing lactic acid, such as bacteria belonging to the genera Lactobacillus, Lactococcus and Bifidobacterium.

The term “probiotic microorganism” refers to a microorganism that form at least a part of the transient or endogenous flora and thereby exhibit a beneficial prophylactic and/or therapeutic effect on the host organism.

The term “prebiotic” are non-digestible food ingredients that stimulate the growth and/or activity of bacteria in the digestive system which are beneficial to the health of the body.

The term “synbiotic” refer to nutritional supplements combining probiotics and prebiotics in a form of synergism, hence synbiotic.

In this specification, unless otherwise specified, “a” or “an” means “one or more”.

### EXAMPLE 1

### Recipe of Organic Bee Pollen Cultivation Media

The invention relates to a product composed of honey, bee bread, bee pollen, pollen and water, at least two of the mentioned ingredients combined. A novel technical description of a superior bacterial media is displayed. The nutrients in pollen are made available together with a perfect mix of carbohydrates from nectar and honey for the cultivation of the honey bee specific LAB.

The best case scenario for LAB cultivation is achieved as follows: A mix of different bee pollen made separately of pollen was obtained from an apiary from colonies maintained using standard beekeeping practices.

Bee pollen pellets were collected from individual bee's legs by pollen traps at the entrance of the bee hive by the beekeeper as soon as foragers returned to the hive. 150.0 g of bee pollen was mixed with 850 g water. The mix was left in room temperature for 2 hours letting the pollen grain burst due to the osmotic shock. The mix were then filtered off the large pollen particles and furthermore centrifuged. The supernatant were filtered from microorganisms originating from the flowers. The filtered product represents the novel bee pollen media used for the LAB cultivation.

### EXAMPLE 2

### Growth of Honey Bee Specific Lactic Acid Bacteria in Novel Media

When the novel bee pollen media is used for cultivation it enhances the bacterial growth of bee specific LAB compared to when grown on previously known and currently purchasable bacterial growth media (MRS) produced for this purpose. The superior growth is achieved even if the MRS media is supplemented and holds the same amount of sugars. A said bee specific LAB strain culture grows much faster meaning that its multiplication time or doubling time is reduced markedly. Also the cultivation end product is greater meaning that the number of cfu is much higher when the cultivation is terminated after growth on the “bee products” media in comparison with the MRS media (FIG. 1).

When the honey stomach LAB were cultivated previously on MRS this media was enforced with fructose and L-cystein which resulted in much better growth in general. The novel bee pollen media contains not only cysteine and fructose naturally but much more of important nutrients for the honey stomach such as proteins, lipids, linoleic acids, unsaturated fatty acids, carbohydrates, fibres, vitamins B1, B2, B3, B5, B6, B9, C and E, minerals such as Copper, Magnesium, Zinc, Potassium and Sodium, polyphenols and flavonoides such as Kaempferol-3.0-glucoside, Isorhamnetine-3.0-glucoside, Rutin, luteoline-7-glucoside, phytosterols, aminoacids such as Threonine, Valine, Methionine, Isoleucine, Leucine, Phenylalanine, Lysine, Tryptophan and Cysteine.

### EXAMPLE 3

### Superior Antimicrobial Properties

When LAB from a bee honey stomach was cultivated on the bee pollen media as in example 1 and 2 their antimicrobial properties were enhanced. The honey stomach LAB have been tested In vitro on traditional agar plates. The LAB were cultivated both separately and together as a flora specific for one bee species. The cultivation method used for the LAB was bee pollen media agar plates and the pathogenic or food spoiling microorganisms were cultivated as an over layer with their respective growth media on top of the LAB.

The food spoiling bacteria from flowers tested are visualized in table 2. The bee larvae pathogenic bacteria and the human pathogenic bacteria tested are visualized in table 3.

In every case where the entire LAB flora from the honey stomach of one honey bee species were tested the pathogenic or food spoiling microorganisms in table 2-3 were strongly inhibited or killed and cells were lysed. When the LAB were tested separately it always differed between test organism how many of the LAB that were effective, everything between 1 to all of the 13. The results showed a less strong result when the LAB were tested alone. The results showed that they have different individual qualities meaning that they produce different antimicrobial substances that make the LAB flora stronger when all are together working as a unit. The results are confirmed with LAB from the bee Apis mellifera via individual substance.

When the tests were repeated with the cultivation of the LAB on MRS media instead of on the novel bee pollen media, less inhibition was displayed and the LAB grew less fast as demonstrated in example 2. These results show clearly that the LAB need their natural nutrients to be able to grow fast, build up new strong cells and produce an efficient amount of antimicrobial substances.

LAB floras from 12 different bees were tested. From the honey bee genera Apis all the 9 recognized species were included which is A. mellifera, A. cerana, A. koshevnikovi, A. nuluensis, A. florea, A. adreniformis, A. laboriosa, A. dorsata and A. nigrocincta. The LAB flora of the stingless bee species Meliponula bocandei and Melipona bechii and Trigona spp. were also included. All of the LAB floras from the different bees included possess a similar LAB flora with more than 10 different species of Lactobacillus sp. and Bifidobacterium sp. as in the case of Apis mellifera displayed in table 1. Most of them are even novel bacterial species of these two genera as their sequences only resembled the closest known taxum with 95-97% that is generally used to define novel a species. The results from the study of the entire identified LAB in said honey bee species will be published 2011 in a scientific journal with the title “Symbiosis between lactic acid bacteria and honey bees”.

TABLE 1
The entire honey stomach LAB flora isolated from the
honey bee species Apis mellifera. Numbers in parenthesis are
GeneBank accession numbers. The bacterial strains listed in table 1 were
deposited at the BCCM/LMG Bacteria Collection in Belgium in
accordance with international deposits under the Budapest Treaty.

| | | Accession |
|---|---|---|
| | | number |
| Bacteria | ain | BCCM/LMG |
| Lactobacillus kunkeei | Fhon2 | LMG P-23987 |
| Lactobacillus sp. | Hon2 | LMG P-24091 |
| Lactobacillus sp. | Biut2 | LMG P-24094 |
| Lactobacillus sp. | Hma2 | LMG P-24093 |
| Lactobacillus sp. | Hma8 | LMG P-24092 |
| Lactobacillus sp. | Bma5 | LMG P-24090 |
| Lactobacillus sp. | Hma11 | LMG P-24612 |
| Lactobacillus sp. | Bin4 | (EF187245) |
| Lactobacillus sp. | Fhon13 | (HM534758) |
| Bifidobacterium sp. | Bin7 | LMG P-23986 |
| Bifidobacterium sp. | Hma3 | LMG P-23983 |
| Bifidobacterium sp. | Bin2 | LMG P-23984 |
| Bifidobacterium sp. | Bma6 | LMG P-23985 |

TABLE 2
Bacteria and yeast isolated from 15 different flowers that bees
forage on. Genera (first word), species (black).
Test-Organisms

Bacteria
Agreia pratensis
Acinetobacter johnsonii
Asaia siamensis
Bacillus simplex
Brenneria quercina
Carnobacterium gallinarum
Cedecea davisae
Curtobacterium flaccumfaciens
Frigoribacterium faeni
Frondihabitans australicus
Fructobacillus pseudoficulneus, persicina, rhapontici, fructosus and
tasmaniensis
Kocuria marina
Lactobacillus paracollinoides, lindneri and concavus
Leuconostoc pseudomesenteroides, mesenteroides, lactis, fallax and
holzapfelii
Microbacterium hydrocarbonoxydans, hominis and profundi
Micrococcus luteus
Pantoea agglomerans and ananatis
Pedobacter heparinus
Plantibacter flavus
Pseudomonas flectens, cannabina, veronii, cichorii, rhizosphaerae,
graminis, umsongensis, koreensis, abietaniphila, agarici and trivialis
Raoultella planticola
Rhodococcus erythropolis and globerulus
Sanguibacter inulinus
Serratia grimesii
Sphingomonas aurantiaca
Variovorax boronicumulans
Xanthomonas arboricola
Yersinia kristensenii
Yeast
Aureobasidium pullulans
Candida rancensis
Crinula caliciiformis
Cryptococcus wieringae
Debaryomyces hansenii var. hansenii and maramus
Hormonema aff. prunorum
Metschnikowia reukaufii and pulcherrima
Rhodotorula fujisanensis
Saccharomycete sp.

TABLE 3
The human pathogenic bacteria and bee larval pathogenic bacteria
are displayed. All of the tested wound bacteria were highly resistant
or multi resistant against penicillin and antibiotics.
Test-Organisms

Food pathogens
Escherichia coli
Listeria inocua
Enterococcus fecalis
Staphylococcus aureus
Bacillus cereus
Human wound pathogens
Enterococcus faecalis
Staphylococcus aureus
Staphylococcus aureus
(MRSA)
Klebsiella aerogenes
Pseudomonas aeruginosa
Enterobacter cloacae
Citrobacter freundii
Serratia narcescens
Escherichia coli
Klebsiella oxytoca
Acinetobacter sp.
Human pathogens
Salmonella typhimurium
Streptococcus
pyogenes
Klebsiella pneumoniae
Clostridium difficile
Bacillus anthracis
Burkholderia
pseudomallei
Burkholderia mallei
Brucella abortus
Brucella melitensis
Propionibacterium
acnes
Bee larval pathogens
Melissococcus plutonius
Paenibacillus larvae

The bacterial growth, according to example 2, is significantly enhanced which not only leads to a substantially higher yield of produced nutrients and antimicrobial substances. The advantage of being numerically superior in the microbial world is equally important. When the bacteria with enhanced antimicrobial properties according to the present invention is administered to bees, to their larvae or to humans together with this media fresh the antimicrobial/probiotic effects are strengthened by the bacterial growth enhancement, a formulation called synbiotic.

### EXAMPLE 4

### Synbiotic Product Base

Both honey/nectar and pollen can be regarded as prebiotic and the LAB can be regarded as probiotic. When combined in a product we have a synbiotic food or feed which enhances the health benefits of the LAB.

The following recipe is performed to create a base of future synbiotic foods or feeds.

The recipe in example 1 is used to create a bee pollen media for the growth of the LAB enhancing their growth, growth rate and the production of their antimicrobial substances. In the recipe in the present example (example 5) the LAB are firstly cultivated separately with the bee pollen media made by the recipe in example 1. After that the bacteria are put together and then freeze dried together with a freshly made bee pollen media according to example 1.

The product is a synbiotic base with the LAB together with all of the essential nutrients preserved from the bee pollen media. The only substance excluded by the freeze drying process is water. When the base is used for example in a bee probiotic it is mixed with different important sugars as glucose, fructose, mannose, sucrose etc given the bees as a feed. But already in the synbiotic base the following sugars are found naturally from the pollen or from the nectar or honey that the bees make their bee pollen with: sucrose, glucose, fructose, mannose, arabinose, xylose, disaccharides, maltose, melibiose, raffinose, melezitose, stachyose and the sugar alcohol sorbitol.

The base can also be included in all sorts of human functional food. Example 5:

Phenotypic Properties of LAB Enhanced with Bee Pollen Media

The LAB that resides in the honey stomach are completely dependent on the nectar and pollen the bees gather and coevolved to use the nutrients in their natural environment. The Lab flora composed of 13 strains function together as a bioreactor that produce varying substances. When said LAB is grown on a combination of “bee products” (the bee pollen media) the availability of the right nutrients that these bacteria have coevolved with is complete and it is a much better growth media than when grown on previously known and currently purchasable bacterial growth media even if supplemented.

The enhanced bacterial growth and growth rate are also very important factors regarding their microbial defence numerically.

Not only the production of classical LAB antimicrobial substances is enhanced but also other substances and nutrients that enhance both the defence system among the honey stomach LAB and the defence system (immune system) among bees and humans and the combination of both since the LAB lives in symbiosis within their host.

The substances that the LAB can produce are depending on the sugars, fatty acids, vitamins, minerals and proteins present in nectar and pollen. The composition of varying sugars (hexoses, pentoses, oligosaccharides, Di-saccharides, monosaccharides), fatty acids (Linoleic acid and a-Linolenic acid), vitamins, proteins, in the pollen/nectar medium result in:
The production of certain B vitamins critical for humans, bee larvae and bees.
A mixture of sugars that enhance the osmotic pressure for the LAB in the presence of fatty acids result in a more stable and antimicrobial membrane by including structures as Exopolysaccharides (EPS), sialic acid, Polyprenols and hyaluronic acid in their membranes. These structures make the LAB modulate the immune system in humans and bees, adhesive to tissues and able to compete against pathogens by producing antimicrobial subtances.
The presence of the monosaccharide xylose results in the production of Xylitol by the LAB. Xylitol is involved in wound management in which the LAB can counteract biofilm formation of pathogens in wounds and anticipate wound healing.
Another monosaccharide, mannose, affects the production of the bacterial membrane in terms of inclusion of sialic acid on the membranes of these LAB making them and their membranes more adhesive to tissues.
Besides their common metabolites, the Lab are able to produce the following substances: 3-hydroxy fatty acids such as Phenyllactic acid (=PLA, substance against molds and yeast), 3-methylbutanal, Benzene, Methylbenzene, Octane, Ethylbenzene, m+p-xylen, o-xylen, Nonane, Hydrogen peroxide (antibacterial), Formic acid (involved in wound healing), Glycerol (biofilm formation and adhesion), Acetoin (antimicrobial), Exopolysaccharides (biofilm formation and adhesion structure) and enzymes (lysis of pathogen cells) etc.

The bees LAB are, when grown on the bee pollen media, able to better inhibit or kill other micoorganisms such as bee pathogens and microorganisms that spoil bee food or feed products. Since the micoorganisms that spoil human food products and the human pathogenic microorganisms are related to the equivalent microorganisms among bees the bee LAB also inhibits or kill these microorganisms efficiently.

1. A method of enhancing antimicrobial properties of a bacterial strain, the method comprising cultivating the bacterial strain in a medium comprising water, and at least one component chosen from bee pollen, pollen, and bee bread, wherein the bacterial strain has the ability to be viable for at least 8 days in a 65% by weight sugar solution.

2. The method according to claim 1, wherein said at least one bacterial strain is chosen from the genus Lactobacillus and the genus Bifidobacterium.

3. The method according to claim 1, wherein the bacterial strain can be isolated naturally from the honey producing tract of at least one bee, wherein the honey producing tract of a bee consists of the trunk, mouth, esophagus and honey sac.

4. The method according to claim 1, wherein said bacterial strain is chosen from Lactobacillus strain Biut2 (LMG P-24094), Lactobacillus strain Hma2 (LMG P-24093), Lactobacillus strain Hma8 (LMG P-24092), Lactobacillus strain BmaS (LMG P-24090), Lactobacillus strain Hon2 (LMG P-24091) said strains being deposited at BCCM/LMG Bacteria Collection in Belgium on 3 Apr. 2007, Bifidobacterium strain Bin7 (LMG P-23986), Bifidobacterium strain Hma3 (LMG P-23983), Bifidobacterium strain Bin2 (LMG P-23984), Bifidobacterium strain Bma6 (LMG P-23985) and Lactobacillus kunkeei Fhon2 (LMG P-23987), said strains being deposited at BCCM/LMG Bacteria Collection in Belgium on 15 Jan. 2007 and Hma11 (LMG P-24612) deposited at BCCM/LMG Bacteria Collection in Belgium on Apr. 28, 2008.

5. The method according to claim 1, wherein the bacterial strain originates from human or animal.

6. A modified bacterial strain produced according to claim 1.

7. A modified bacterial strain produced according to claim 1 for use in the treatment and/or prevention of throat infections.

8. A modified bacterial strain produced according to claim 1 for use in the treatment of a wound.

9. A modified bacterial strain for use according to claim 8, wherein the treatment of a wound includes promotion of wound healing.

10. A pharmaceutical composition comprising a modified bacterial strain according to claim 6 and a pharmaceutically acceptable carrier and/or diluent.

11. Feed or food product comprising a modified bacterial strain according to claim 6.

12. A cultivation medium for lactic acid bacteria comprising at least one component chosen from bee pollen, pollen, and bee bread, and another component chosen from water.

13. The cultivation medium according to claim 12, wherein said bee pollen, pollen, and bee bread have been obtained from bee pollen grains.

14. A synbiotic comprising a cultivation medium according to claim 12, and at least one strain of lactic acid bacteria.

15. The synbiotic according to claim 14, wherein said at least one strain of lactic acid bacteria is chosen from the genus Lactobacillus and the genus Bifidobacterium.

16. The method according to claim 1, wherein the bacterial strain has the ability to be viable for at least 8 days in a 70% by weight sugar solution.

17. The method according to claim 1, wherein the medium further comprises honey and/or nector.

18. The cultivation medium according to claim 12, wherein the cultivation medium further comprises honey and/or nector.

19. The cultivation medium according to claim 18, wherein the honey and/or nector have been obtained from bee pollen grains.
